(19) United States
(12) Patent Application Publication
(10) Pub. No.: US 2004/0211657 A1
(43) Pub. Date: Oct. 28, 2004

(54) METHOD OF PURIFYING 2,6-XYLENOL AND METHOD OF PRODUCING POLY(ARYLENE ETHER) THEREFROM

(76) Inventor: Hugo Gerard Eduard Ingelbrecht, Essen (BE)

Correspondence Address: CANTOR COLBURN, LLP, 55 GRIFFIN ROAD SOUTH, BLOOMFIELD, CT, 06002

(21) Appl. No.: 10/249,462

(22) Filed: Apr. 11, 2003

(51) Int. Cl. B01D 3/42; C07C 27/26; C07C 29/74

(52) U.S. Cl. 203/1; 203/3; 203/74; 203/75; 203/78; 568/913

(57) ABSTRACT

A method of purifying 2,6-dimethylphenol to remove the odorous impurities includes distilling a crude mixture to yield a first light fraction enriched in 2,6-dimethylphenol, and a first heavy fraction enriched in 2,4,6-trimethylanisole, and distilling the first light fraction to yield a second light fraction, and a second heavy fraction enriched in 2,6-dimethylphenol. The concentration of 2,4,6-trimethylanisole in the second heavy fraction is less than 50% of the concentration of 2,4,6-trimethylanisole in the mixture. The purified 2,6-dimethylphenol is useful for preparing low-odor poly(arylene ether) resins.

### BACKGROUND

Poly(arylene ether) resins are widely used and valued for their heat resistance and physical properties including impact strength and stiffness. However, a variety of odorous impurities that may be present in poly(arylene ether) resins have discouraged their adoption for odor-sensitive applications such as containers for food, cosmetics, and pharmaceuticals.

One approach to reducing the odor of poly(arylene ether) resins is to increase the purity of the alkyl phenols from which they are prepared. U.S. Pat. No. 3,483,094 to Parisse describes a method of purifying alkyl phenols by azeotropically distilling them with C₁₀-C₁₃ alkenes. U.S. Pat. No. 3,517,072 to Moroni et al. describes purification of alkyl phenols by countercurrent washing with an aqueous alkali metal hydroxide. Great Britain Patent No. 1,165,122 to Leston describes a method of purifying 2,6-dimethylphenol from a mixture containing monomethylphenols by selectively forming methyl ethers from the monomethylphenols before distilling the mixture. All of the above methods require the introduction of additional materials. There remains a need for an improved and simplified method of reducing the odor of 2,6-dimethlphenol and its polymerization products.

### BRIEF SUMMARY

The above-described and other drawbacks are alleviated by a method of purifying 2,6-dimethylphenol, comprising: distilling a mixture comprising 2,6-dimethylphenol and 2,4,6-trimethylanisole to yield a first light fraction enriched in 2,6-dimethylphenol, and a first heavy fraction enriched in 2,4,6-trimethylanisole; and distilling the first light fraction to yield a second light fraction, and a second heavy fraction enriched in 2,6-dimethylphenol; wherein the weight percent of 2,4,6-trimethylanisole in the second heavy fraction is less than 50% of the weight percent of 2,4,6-trimethylanisole in the mixture.

Other embodiments, include a method of producing a low-odor poly(arylene ether), are described in detail below.

### BRIEF DESCRIPTION OF DRAWINGS

FIG. 1 is a diagrammatic view of a distillation apparatus suitable for performing the purification method.

FIG. 2 is a diagrammatic view of solvent recycling apparatus used in poly(arylene ether) synthesis.

### DETAILED DESCRIPTION OF THE PREFERRED EMBODIMENTS

One embodiment is a method of purifying 2,6-dimethylphenol, comprising: distilling a mixture comprising 2,6-dimethylphenol and 2,4,6-trimethylanisole to yield a first light fraction enriched in 2,6-dimethylphenol, and a first heavy fraction enriched in 2,4,6-trimethylanisole; and distilling the first light fraction to yield a second light fraction, and a second heavy fraction enriched in 2,6-dimethylphenol; wherein the weight percent of 2,4,6-trimethylanisole in the second heavy fraction is less than 50% of the weight percent of 2,4,6-trimethylanisole in the mixture.

One method of purifying 2,6-dimethylphenol (sometimes called 2,6-xylenol) comprises a two-step distillation in which a mixture is distilled to yield a first heavy fraction enriched, relative to the mixture, in 2,6-dimethylphenol, and a first light fraction enriched in lower-boiling impurities. The first heavy fraction is then subjected to a second distillation to yield a second light fraction further enriched in 2,6-dimethylphenol and a second heavy fraction enriched in higher-boiling impurities. While the 2,6-dimethylphenol obtained by this process can be used to form poly (arylene ether) resins suitable for may purposes, the resins sometimes have odors that make them undesirable for more esthetically demanding applications, such as packaging for food products. The present inventors have discovered that odorous impurities in 2,6-dimethylphenol—and poly(arylene ether) resins synthesized therefrom—may be greatly reduced by purifying crude 2,6-dimethylphenol using a method comprising a distillation of the crude mixture to yield a first light fraction enriched in 2,6-dimethylphenol and a first heavy fraction enriched in 2,4,6-trimethylanisole, and a distillation of the first light fraction to yield a second light fraction enriched in low-boiling impurities and a second heavy fraction further enriched in 2,6-dimethylphenol.

There is no particular limitation on the source of the mixture comprising 2,6-dimethylphenol and, 2,4,6-trimethylanisole. Methods of preparing 2,6-dimethylphenol that may generate 2,4,6-trimethylanisole as an impurity are described, for example, in U.S. Pat. Nos. 4,661,638 to Battista et al., 4,933,509 to Warner, 5,097,079 to Bennett, Jr., 5,912,391 to Barnhart et al., 6,291,724 to Braat, and 6,395,871 to Watson et al.

The mixture subjected to distillation may generally comprise at least about 10 weight percent of 2,6-dimethylphenol. In a preferred embodiment, the mixture comprises at least about 50, more preferably at least about 55, still more preferably at least about 60 weight percent of 2,6-dimethylphenol. The compound mixture also comprises the odorous impurity 2,4,6-trimethylanisole, which may be present at up to 5 weight percent or more. Generally, the mixture comprises at least 20 parts per million by weight of 2,4,6-trimethylanisole. The mixture may also comprise other odorous impurities, including 2,6-dimethylcyclohexanone, which may be present at about 100 to about 10,000 parts per million by weight.

The distillation of the mixture yields a first light fraction enriched in 2,6-dimethylphenol, and a first heavy fraction enriched in 2,4,6-trimethylanisole. “Enriched” means that the concentration of the specified compound is higher in the distillate (i.e., the respective distillation fraction) than it is in the feed of the particular distillation column. For example, “a first heavy fraction enriched in 2,4,6-trimethylanisole” means that the first heavy fraction has a greater weight percent of 2,4,6-trimethylanisole than the mixture distilled to yield the first light fraction and the first heavy fraction.

Apparatus for lab-scale, pilot-scale, and industrial-scale distillation is known in the art. To maximize the separation of 2,6-dimethylphenol from 2,4,6-trimethylanisole and other impurities including 2,3-dimethylphenol, phenol, 2-methylphenol, 2,4-dimethylphenol, 2,4,6-trimethylphenol, 2,6-dimethylcyclohexanone, anisole, and lower methylated anisoles, it may be preferred to distill the mixture with a column having at least 35 theoretical plates. It may also be preferred to distill the mixture with a reflux ratio of about 2.5 to about 15, more preferably about 3 to about 10, where reflux ratio is defined as the ratio of the mass of the flow returned back to the column as reflux to the mass of the flow of top product removed as distillate. It may also be preferred to distill the mixture with a ratio of bottom flow to feed of about 0.02 to about 0.10, more preferably about 0.02 to about 0.06, where the “bottom flow” is defined as the flow out the bottom of the column, and the “feed” is defined as the flow of mixture to the column.

The method further includes distilling the first light fraction to yield a second light fraction, and a second heavy fraction enriched in 2,6-dimethylphenol. The second heavy fraction may be drawn from among the lower 10% of theoretical plates in the second distillation column. The weight percent of 2,4,6-trimethylanisole in the second heavy fraction is less than 50%, preferably less than 30%, more preferably less than 20% of the weight percent of 2,4,6-trimethylanisole in the mixture. In one embodiment, the second heavy fraction comprises less than or equal to 500, preferably less than or equal to 300, more preferably less than or equal to 100 parts per million by weight of 2,4,6-trimethylanisole.

In one embodiment, the mixture comprises 2,6-dimethylcyclohexanone, and the second heavy fraction comprises less than 50%, preferably less than 15%, more preferably less than 8% of the weight percent of 2,6-dimethylcyclohexanone in the mixture. In a preferred embodiment, the second heavy fraction comprises less than 400 parts per million by weight, more preferably less than 200 parts per million by weight of 2,6-dimethylcyclohexanone.

One advantage of the method is its ability to reduce the odorous impurities in 2,6-dimethylphenol without sacrificing the yield of 2,6-dimethylphenol. Specifically, the second heavy fraction may comprise at least about 90, preferably at least about 95, more preferably at least about 99.5, still more preferably at least about 99.8 percent of the 2,6-dimethylphenol present in the mixture.

To maximize the separation of 2,6-dimethylphenol and lower-boiling impurities, it may be preferred to distill the first light fraction with a column having at least 25 theoretical plates. In a preferred embodiment, distillation of the first light fraction comprises distilling with at least 35, preferably at least 45, more preferably at least 55 theoretical plates. It may be preferred to introduce the first light fraction above 25 percent of the theoretical plates and below about 80 percent of the theoretical plates, more preferably above 35 percent of the theoretical plates and below about 70 percent of the theoretical plates. In a preferred embodiment, distilling the first light fraction comprises distilling with a reflux ratio of about 10 to about 40, more preferably about 15 to about 30. In another preferred embodiment, distilling the first light fraction comprises distilling with a ratio of bottom flow to feed of about 0.50 to about 0.99, preferably about 0.70 to about 0.99.

The method provides a high yield of 2,6-dimethylphenol based on the 2,6-dimethylphenol content of the mixture. In one embodiment, the second heavy fraction comprises at least about 90%, preferably at least 95%, more preferably at least 98%, still more preferably at least 99% of the 2,6-dimethylphenol present in the mixture.

The method yields 2,6-dimethylphenol with substantially reduced concentrations of odorous impurities. Still further reductions in odorous impurities may be achieved by melt crystallizing the second heavy fraction. Techniques and apparatus for fractional melt crystallizing are known in the art and described, for example, in U.S. Pat. Nos. 3,621,664 to Saxer, and 5,243,093 to Kissinger et al. In melt crystallization, the temperature is lowered gradually until the temperature is somewhat below the melting point of the desired substance. In some cases the composition may have to be heated above the melting temperature of the desired substance and then brought down below the freezing point. Ideally, the desired component crystallizes onto the surface of the vessel holding the melt composition. The theory of the fractional melt crystallization is that the desired component preferentially is crystallized out from the melt while the undesired impurities remain in their liquid state or are entrapped in the crystalline medium to a limited extent. In a multiple stage fractional melt crystallization, the crystalline desired component's purity is upgraded in each successive stage, through the phases of crystallization, partial melting (sweating), and total melting. A preferred apparatus to carry out the fractional melt crystallization is referred to as the “Sulzer” melt crystallization apparatus. This is a falling film dynamic crystallizer, which is obtained from Sulzer Canada, Inc., a subsidiary of Sulzer Brothers, Ltd., Switzerland. It may be preferred to carry out at least two cycles of melt crystallization. Cycles, stages, and conditions of melt crystallization are further described and illustrated in Example 4, below.

One embodiment is a method of purifying 2,6-dimethylphenol, comprising: distilling a mixture comprising 2,6-dimethylphenol, 2,6-dimethylcyclohexanone, and 2,4,6-trimethylanisole to yield a first light fraction enriched in 2,6-dimethylphenol and 2,6-dimethylcyclohexanone, and a first heavy fraction enriched in 2,4,6-trimethylanisole; and distilling the first light fraction to yield a second light fraction enriched in 2,6-dimethylcyclohexanone, and a second heavy fraction enriched in 2,6-dimethylphenol; wherein the weight percent of 2,4,6-trimethylanisole in the second heavy fraction is less than 50% of the weight percent of 2,4,6-trimethylanisole in the mixture; and wherein the weight percent of 2,6-dimethylcyclohexanone in the second heavy fraction is less than 50% of the weight percent of 2,6-dimethylcyclohexanone in the mixture.

Another embodiment is a method of purifying 2,6-dimethylphenol, comprising: distilling a mixture comprising at least 60 weight percent 2,6-dimethylphenol, at least 100 parts per million by weight 2,6-dimethylcyclohexanone, and at least 20 parts per million by weight 2,4,6-trimethylanisole to yield a first light fraction enriched in 2,6-dimethylphenol and 2,6-dimethylcyclohexanone, and a first heavy fraction enriched at least two-fold in 2,4,6-trimethylanisole; and distilling the first light fraction to yield a second light fraction enriched in 2,6-dimethylcyclohexanone, and a second heavy fraction enriched in 2,6-dimethylphenol; wherein the second heavy fraction comprises at least about 95 percent of the 2,6-dimethylphenol present in the mixture; and wherein the weight percent of 2,4,6-trimethylanisole in the second heavy fraction is less than 50% of the weight percent of 2,4,6-trimethylanisole in the mixture.

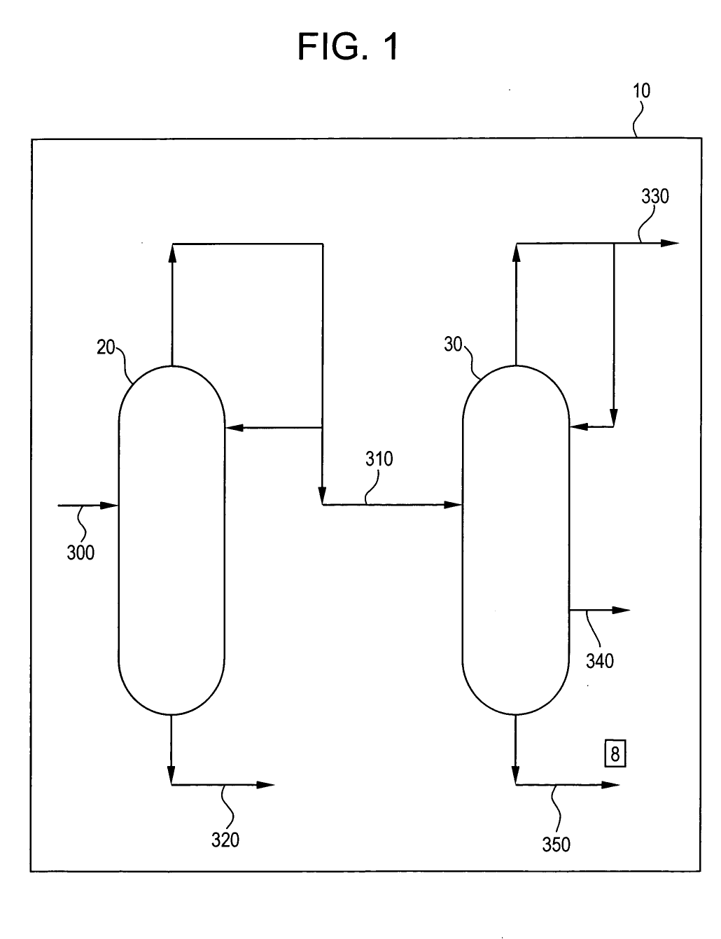

FIG. 1 is a diagrammatic view of a distillation apparatus 10 suitable for performing the purification method. 2,6-Dimethylphenol-containing mixture 300 is introduced to a first distillation column 20, where it is separated to yield a first light fraction 310 enriched in 2,6-dimethylphenol and a first heavy fraction 320 enriched in 2,4,6-trimethylanisole. The first light fraction 310 is introduced to a second distillation column 30, where it is separated into a second light fraction 330 and a second heavy fraction 340. A second ultraheavy fraction 350, corresponding to the bottom flow, may also be separated. The second light fraction 340 is a highly purified 2,6-dimethylphenol that may be used, with or without further purification, for odor-sensitive applications.

The low-odor 2,6-dimethylphenol is particularly useful for synthesizing a low-odor poly(arylene ether) comprising a plurality of 2,6-dimethyl-1,4-phenylene units. Thus, one embodiment is a method of producing a low-odor poly(arylene ether), comprising: purifying 2,6-dimethylphenol by one of the above methods and polymerizing a dihydric phenol comprising at least 10 weight percent of 2,6-dimethylphenol obtained from the second heavy fraction in an aromatic hydrocarbon solvent to form a poly(arylene ether); and isolating the poly(arylene ether). Methods for synthesizing poly(arylene ether) homopolymers and copolymers, including catalysts and aromatic hydrocarbon solvents, are described, for example, in U.S. Pat. Nos. 3,306,874 and 3,306,875 to Hay, and 4,011,200 and 4,038,343 to Yonemitsu et al. Aromatic hydrocarbon solvents include, for example, C₆-C₁₈ aromatic hydrocarbons, such as toluene, xylenes, ethylbenzene, and the like, and mixtures thereof. By utilizing low-odor 2,6-dimethylphenol prepared by the above methods, poly(arylene ether) resins prepared from the 2,6-dimethylphenol may comprise less than 50, preferably less than 40, more preferably less 30, even more preferably less than 10 parts per million by weight of 2,4,6-trimethylanisole.

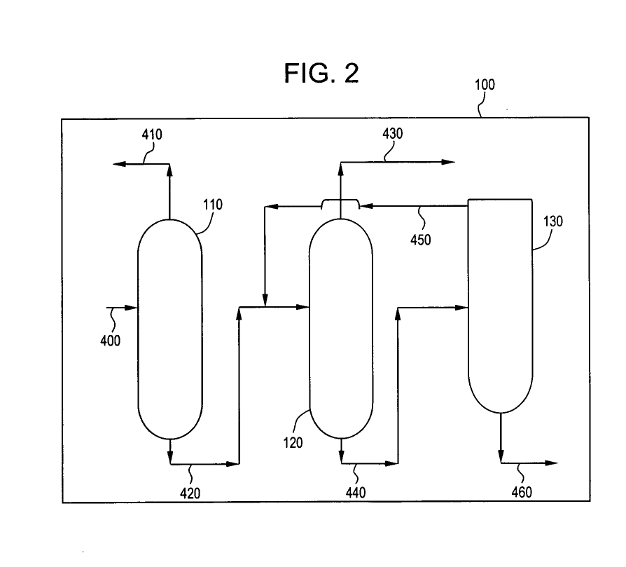

In the synthesis of poly(arylene ether) resins from low-odor 2,6-dimethylphenol, it is often desirable to recover the aromatic hydrocarbon reaction solvent and recycle it to the reactor. However, known methods for recycling aromatic hydrocarbon solvent may increase the concentration of odorous impurities in the poly(arylene ether) resin. FIG. 2 is a diagrammatic view of solvent recycling apparatus 100 suitable for use in preparing a low-odor poly(arylene ether) resin. Isolation of product poly(arylene ether) by precipitation and filtration generates a solvent-antisolvent mixture 400 (e.g., a mixture of toluene as solvent and methanol as antisolvent) that is introduced to a third distillation column 110. The third distillation column 110 separates the solvent-antisolvent mixture 400 into an antisolvent-rich third light fraction 410 and a solvent-rich third heavy fraction 420. The third heavy fraction 420 is directed to an evaporation column 120 (evaporation columns are described, for example, in U.S. Pat. No. 3,755,088 to Osdor), where it is separated into a solvent-rich fourth light fraction 430 and a fourth heavy fraction 440. The fourth heavy fraction 440, which typically contains about 2 to about 5 weight percent solids, is then further concentrated in a wiped film evaporator 130 to yield a solvent-containing evaporated fraction 450 and a concentrated fraction 460. The evaporated fraction 450 may be recycled to the inlet of the evaporation column 120. The solvent recycling process is described below in greater detail using toluene as the aromatic hydrocarbon solvent.

It has now been found that by controlling the solids content of the concentrated fraction 450 to less than 50 weight percent solids, preferably less than 30 weight percent solids, more preferably less than 20 weight percent solids, still more preferably less than 15 weight percent solids, the odor components are enriched in the concentrated fraction 460 and thereby separated from the recycled solvent stream. Percent solids is determined by depositing the solution of interest on a pre-weighed aluminum disk, heating the disk on an electrical hot plate for about 30 minutes to evaporate the solution to dryness, and reweighing the disk to determine the weight of non-volatile material (i.e., solids). Wiped film evaporators and methods for their use are known in the art and described, for example, in U.S. Pat. Nos. 3,687,983 to Widmer et al., 3,695,327 to Widmer, and 4,054,485 to Linder et al.

The odor of the poly(arylene ether) may be further reduced by distilling the aromatic hydrocarbon solvent top product from the wiped film evaporator in separate distillation step. A suitable method of distilling the aromatic solvent immediately prior to recycling it to the reactor is described in U.S. Pat. No. 4,906,700 to Banevicius. The distilled aromatic hydrocarbon solvent will preferably comprises less than 50 percent of the 2,4,6-trimethylanisole present prior to distillation. The concentration of 2,4,6-trimethylanisole in the distilled aromatic hydrocarbon solvent will preferably be less than 30 parts per million by weight.

The invention is further illustrated by the following non-limiting examples.

### WORKING EXAMPLES

### Example 1

A mixed alkylated phenol feed stream of 5.6 ton/hr was supplied to a position corresponding to plate 37 of a distillation column containing 84 theoretical plates. The top product (distillate) flow rate was 5.2 ton/hr and bottom flow rate was 0.7 ton/hr. The reflux ratio was 4.9. Analysis of the bottom product and top product gave the results in Table 1.

TABLE 1

| | Bottom product | Top product |
|---|---|---|
| 2,4,6-trimethylanisole (wt %) | 0.892 | 0.012 |
| 2,6-dimethylcyclohexanone (wt %) | 0.0042 | 0.0475 |
| 2,6-dimethylphenol (wt %) | 2.66 | 69.6 |

The calculated % removal of TMA (ratio of the mass flow rate in the bottom stream vs. the column feed) amounts to 85% and for 2,6-dimethylcyclohexanone amounts to <1%.

### Example 2

The top product from Example 1 was fed at a flow rate of 4.8 ton/hr to a position corresponding to plate 57 of a second distillation column containing 84 theoretical plates. The top product flow rate, corresponding to the second light fraction, was 1.3 ton/hr and 2,6-dimethylphenol-enriched second heavy fraction flow rate of 3.5 ton/hr. The reflux ratio was 27.5. Analyses of the top product and the second heavy fraction gave the results in Table 2.

TABLE 2

| | top product | second heavy fraction |
|---|---|---|
| 2,4,6-trimethylanisole (wt %) | 0.0011 | 0.0255 |
| 2,6-dimethylcyclohexanone (%) | 0.16 | 0.017 |
| 2,6-dimethylphenol (%) | 1.30 | 99.85 |

The calculated % removal of 2,4,6-trimethylanisole (ratio of the mass flow rate in the top product stream vs. the column feed) amounts to 1.5% and for 2,6-dimethylcyclohexanone amounts to 78%.

### Example 3

A poly(2,6-dimethylphenylene ether) was prepared by polymerization of 2,6-dimethylphenol in toluene solvent. The toluene solvent was continuously recycled. A feed stream of toluene containing a low concentration of non-volatile low molecular weight poly(2,6-dimethylphenylene ether) oligomers was supplied at a feed rate of 7.15 ton/hr to an evaporation column. The toluene and lower boiling impurities were evaporated and a bottom product was discharged at a flow rate of 510 kg/hr. This bottom stream was subsequently fed to a LUWA® wiped film evaporator to achieve additional vaporization and recovery of toluene with a residual bottom stream percent solids of 5 to 18 weight percent. Concentrations of odorous impurities in the final poly(2,6-dimethylphenylene ether) powder were determined and are present in Table 3. Concentration are expressed in parts per million by weight (ppmw).

TABLE 3

| Odorous impurity | Concentration (ppmw) |
|---|---|
| 2,4,6-trimethylanisole | 32 |
| 2,6-dimethylcyclohexanone | 23 |
| 2,3-dihydrobenzofuran | 72 |

Increasing the percent solids from 5-18 weight percent to 60 weight percent increased the content of odor components by a factor of 4.5.

### Example 4

The example describes purification of 2,6-xylenol by melt crystallization. A sample of molten 2,6-xylenol (14 kg), which had been purified by a distillation process similar to that described in Examples 1 and 2, was fed to a batch melt crystallizer (SULZER®) with a single 12-meter tube for a cycle time of 180 minutes. The batch cycle consisted of 3 steps:

Step 1, Crystallization: The liquid is circulated over the crystallizer tube. At the outside of the tube the cooling medium flows as a thin film. Due to the temperature gradient with the melt, crystals are formed on the tube wall. The molten residue is directed to the residue tank.

Step 2, Partial melting: The crystals along the wall are partially melted to remove impurities eventually occluded in the crystals.

Step 3, Melting: The crystals along the tube walls are melted and directed to the product tank for crystallization in a second cycle.

Analysis results of the feed stream, residue and crystals from cycles 1 and 2 are given in Table 4. The results show that ultra-high purity 2,6-dimethylphenol may be obtained by a combination of distillation and melt crystallization purifications.

TABLE 4

| | | |
|---|---|---|
| Feed | Residue | Crystal product |

Cycle 1

| | | | |
|---|---|---|---|
| 2,6-dimethylphenol (wt %) | 99.879 | 99.614 | 99.978 |
| 2,6-dimethylcyclohexanone (wt %) | <0.001 | 0.005 | <0.001 |
| 2-methylphenol (wt %) | 0.009 | 0.025 | <0.001 |
| meta- and para-cresol (wt %) | 0.020 | 0.064 | 0.010 |
| 2,3-dihydrobenzofuran (wt %) | 0.014 | 0.045 | <0.001 |
| 2,4-dimethylanisole (wt %) | 0.002 | 0.007 | <0.001 |
| 2-ethylphenol (wt %) | 0.020 | 0.060 | 0.003 |
| 2,4,6-trimethylanisole (wt %) | 0.057 | 0.175 | 0.009 |

Cycle 2

| | | | |
|---|---|---|---|
| 2,6-dimethylphenol (wt %) | 99.988 | 99.949 | 100.00 |
| 2,6-dimethylcyclohexanone (wt %) | <0.001 | <0.001 | <0.001 |
| 2-methylphenol (wt %) | <0.001 | 0.004 | <0.001 |
| meta- and para-cresol (wt %) | 0.003 | 0.008 | <0.001 |
| 2,3-dihydrobenzofuran (wt %) | <0.001 | 0.013 | <0.001 |
| 2,4-dimethylanisole (wt %) | <0.001 | <0.001 | <0.001 |
| 2-ethylphenol (wt %) | 0.010 | 0.021 | <0.001 |
| 2,4,6-trimethylanisole (wt %) | 0.006 | 0.024 | <0.001 |

The above examples illustrate that the purification method may be used to obtain high purity 2,6-dimethylphenol, and that poly(arylene ether) resins having low concentrations of odorous impurities may be prepared from the purified 2,6-dimethylphenol.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

All cited patents, patent applications, and other references are incorporated herein by reference in their entirety.

1. A method of purifying 2,6-dimethylphenol, comprising:
distilling a mixture comprising 2,6-dimethylphenol and 2,4,6-trimethylanisole to yield a first light fraction enriched in 2,6-dimethylphenol, and a first heavy fraction enriched in 2,4,6-trimethylanisole; and
distilling the first light fraction to yield a second light fraction, and a second heavy fraction enriched in 2,6-dimethylphenol;
wherein the weight percent of 2,4,6-trimethylanisole in the second heavy fraction is less than 50% of the weight percent of 2,4,6-trimethylanisole in the mixture.

2. The method of claim 1, wherein said distilling a mixture comprises distilling with a column having at least 35 theoretical plates.

3. The method of claim 1, wherein said distilling the first light fraction comprises distilling with a column having at least 35 theoretical plates.

4. The method of claim 1, wherein said distilling a mixture comprises distilling with a reflux ratio of about 2.5 to about 15.

5. The method of claim 1, wherein said distilling a mixture comprises distilling with a ratio of bottom flow to feed of about 0.02 to about 0.10.

6. The method of claim 1, wherein said distilling the first light fraction comprises distilling with a reflux ratio of about 10 to about 40.

7. The method of claim 1, wherein said distilling the first light fraction comprises distilling with a ratio of bottom flow to feed of about 0.50 to about 0.99.

8. The method of claim 1, wherein said distilling the first light fraction comprises distilling with a column having at least 35 theoretical plates and introducing the first light fraction above 35 percent of the theoretical plates and below 70 percent of the theoretical plates.

9. The method of claim 1, wherein the mixture comprises at least 50 weight percent of 2,6-dimethylphenol.

10. The method of claim 1, wherein the mixture comprises about 0.002 to about 0.2 weight percent 2,4,6-trimethylanisole.

11. The method of claim 1, wherein the second heavy fraction comprises less than 300 parts per million by weight of 2,4,6-trimethylanisole.

12. The method of claim 1, wherein the mixture further comprises 2,6-dimethylcyclohexanone, and wherein the weight percent of 2,6-dimethylcyclohexanone in the second heavy fraction is less than 50% of the weight percent of 2,6-dimethylcyclohexanone in the mixture.

13. The method of claim 12, wherein the weight percent of 2,6-dimethylcyclohexanone in the second heavy fraction is less than 200 parts per million by weight.

14. The method of claim 1, wherein the second heavy fraction comprises at least about 90 percent of the 2,6-dimethylphenol present in the mixture.

15. The method of claim 1, further comprising melt crystallizing 2,6-dimethylphenol obtained from the second heavy fraction.

16. A method of purifying 2,6-dimethylphenol, comprising:
distilling a mixture comprising 2,6-dimethylphenol, 2,6-dimethylcyclohexanone, and 2,4,6-trimethylanisole to yield a first light fraction enriched in 2,6-dimethylphenol and 2,6-dimethylcyclohexanone, and a first heavy fraction enriched in 2,4,6-trimethylanisole; and
distilling the first light fraction to yield a second light fraction enriched in 2,6-dimethylcyclohexanone, and a second heavy fraction enriched in 2,6-dimethylphenol;
wherein the weight percent of 2,4,6-trimethylanisole in the second heavy fraction is less than 50% of the weight percent of 2,4,6-trimethylanisole in the mixture; and
wherein the weight percent of 2,6-dimethylcyclohexanone in the second heavy fraction is less than 50% of the weight percent of 2,6-dimethylcyclohexanone in the mixture.

17. A method of purifying 2,6-dimethylphenol, comprising:
distilling a mixture comprising at least 50 weight percent 2,6-dimethylphenol, at least 100 parts per million by weight 2,6-dimethylcyclohexanone, and at least 20 parts per million by weight 2,4,6-trimethylanisole to yield a first light fraction enriched in 2,6-dimethylphenol and 2,6-dimethylcyclohexanone, and a first heavy fraction enriched at least two-fold in 2,4,6-trimethylanisole; and
distilling the first light fraction to yield a second light fraction enriched in 2,6-dimethylcyclohexanone, and a second heavy fraction enriched in 2,6-dimethylphenol;
wherein the second heavy fraction comprises at least about 95 percent of the 2,6-dimethylphenol present in the mixture;
wherein the weight percent of 2,4,6-trimethylanisole in the second heavy fraction is less than 50% of the weight percent of 2,4,6-trimethylanisole in the mixture; and
wherein the weight percent of 2,6-dimethylcyclohexanone in the second heavy fraction is less than 50% of the weight percent of 2,6-dimethylcyclohexanone in the mixture.

18. A method of producing a low-odor poly(arylene ether), comprising:
purifying 2,6-dimethylphenol by a method comprising distilling a mixture comprising 2,6-dimethylphenol and 2,4,6-trimetnylanisole to yield a first light fraction enriched in 2,6-dimethylphenol, and a first heavy fraction enriched in 2,4,6-trimethylanisole; and
distilling the first light fraction to yield a second light fraction, and a second heavy fraction enriched in 2,6-dimethylphenol;
wherein the weight percent of 2,4,6-trimethylanisole in the second heavy fraction is less than 50% of the weight percent of 2,4,6-trimethylanisole in the mixture;
polymerizing a dihydric phenol comprising at least 10 weight percent of 2,6-dimethylphenol obtained from the second heavy fraction in an aromatic hydrocarbon solvent to form a poly(arylene ether); and
isolating the poly(arylene ether).

19. The method of claim 18, wherein the isolated poly(arylene ether) comprises less than 50 parts per million by weight of 2,4,6-trimethylanisole.

20. The method of claim 18, further comprising recovering the aromatic hydrocarbon solvent, wherein recovering the aromatic hydrocarbon solvent comprises distilling the aromatic solvent to yield a top stream and a bottom stream, treating the bottom stream with a wiped film evaporator to yield an evaporated aromatic hydrocarbon solvent, and controlling the solids content in the wiped film evaporator to less than or equal to 50 weight percent.

21. The method of claim 20, further comprising distilling the evaporated aromatic hydrocarbon solvent.

22. The method of claim 21, wherein the evaporated aromatic hydrocarbon solvent prior to distillation comprises 2,4,6-trimethylanisole, and wherein the evaporated aromatic hydrocarbon solvent after distillation comprises less than 50 percent of the 2,4,6-trimethylanisole present prior to distillation.

23. The method of claim 21, wherein the evaporated aromatic hydrocarbon solvent after distillation comprises less than 50 parts per million by weight of 2,4,6-trimethylanisole.
